(19) United States
(12) Patent Application Publication
(10) Pub. No.: US 2017/0336389 A1
(43) Pub. Date: Nov. 23, 2017

(54) SENSOR ASSEMBLIES AND METHODS OF USE

(71) Applicant: ZANSORS LLC, Tysons, VA (US)

(72) Inventors: Shane Trexler, Arlington, VA (US); Abhijit Dasgupta, Germantown, MD (US)

(21) Appl. No.: 15/602,321

(22) Filed: May 23, 2017

Related U.S. Application Data
(60) Provisional application No. 62/340,330, filed on May 23, 2016.

(51) Int. Cl. G01N 33/497; A61B 5/00; G01N 33/487

(52) U.S. Cl. CPC G01N 33/4972; G01N 33/487; A61B 5/742; A61B 5/0004; A61B 5/4845; A61B 5/6831

(57) ABSTRACT

A sensor assembly may comprise a housing, a sensor mounted in the housing, and an open cavity. The sensor may include a sensing element adapted to detect a target and generate an electrical signal in response to detecting the target. The open cavity may be positioned to expose the sensing element to a fluid. The open cavity may comprise a continuous distal perimeter adapted to define a substantially closed volume when the continuous distal perimeter is contacted with a surface.

### CROSS-REFERENCE TO RELATED APPLICATIONS

This application claims priority from pending U.S. Provisional Patent Application 62/340,330, filed on May 23, 2016, the disclosure of which is included by reference herein in its entirety.

### BACKGROUND OF THE INVENTION

The presence of certain substances in fluids can reveal information about the fluids and/or the source of the fluids. For example, physiological conditions can be recognized through detecting substances in bodily fluids such as blood or sweat.

In one example, the presence of alcohol can be detected in bodily fluids. identifying drivers under the influence of alcohol occurs only after alcohol consumption when individuals at risk are already behind the wheel or pulled over at sobriety checkpoints by law enforcement with the use of a breathalyzer test. In 2010, despite education and awareness, 112 million individuals self-reported being impaired while driving their vehicle and 1.2 million individuals were arrested for driving under the influence of alcohol or narcotics. It was reported in 2012 that 10,322 deaths were related to alcohol-impaired driving crashes and the annual cost related to alcohol related incidents totals close to $59 billion. Responsible adults understand the risks and have proper access to alcohol education/resources, which may not be available during consumption, but still make the decision to drive while impaired, not knowing their actual blood alcohol concentration leading to the risk of an accident.

Blood Alcohol Concentration (BAC) education for consumers typically provides information on impairment effects on the body for male or female adults based on the amount of consumption in units and types of alcohol consumed (liquor, beer or wine). Social drinking guidelines would need to be practiced by consumers to restrict BAC levels below 0.08%, but this consumption will vary for individuals based on weight and time of beverages consumed. While consuming alcoholic beverages, consumers would need a readily available device to test blood alcohol levels or breathalyzer to test a sample of breath. Commercially wearable alcohol sensor products are available, but they are typically sold for law enforcement, often obtrusive, and not appealing to the wearer. Alcohol serving bars and restaurants can implement self-testing machines, but these can cost $1200+ and bring a social stigma to consumers when using these machines in public venues. On the other hand, consumers typically have access to smartphones that are readily available and used at bars and restaurants. Connecting a discreet, low profile wearable sensing device through wireless communication to an application on a smartphone could deliver real-time notifications of their BAC

### SUMMARY OF THE INVENTION

Systems and methods described herein include sensor assemblies and methods for using the same. The sensor assemblies may be configured to gather fluids so that they may be analyzed for the presence of targets. For example, a sensor assembly may comprise a housing, a sensor mounted in the housing, and an open cavity. The sensor may include a sensing element adapted to detect a target and generate an electrical signal in response to detecting the target. The open cavity may be positioned to expose the sensing element to a fluid. The open cavity may comprise a continuous distal perimeter adapted to define a substantially closed volume when the continuous distal perimeter is contacted with a surface.

### BRIEF DESCRIPTION OF THE DRAWINGS

The subject matter, which is regarded as the invention, is particularly pointed out and distinctly recited in the claims at the conclusion of the specification. The foregoing and other features and advantages of the invention, in its many aspects, will be readily understood from the following detailed description of aspects of the invention taken in conjunction with the accompanying drawings in which:

FIG. 1 is a perspective view of a sensor assembly according to one aspect of the invention.

FIG. 2 is a bottom view of the sensor assembly shown in FIG. 1.

FIG. 3 is a right side elevation view of the sensor assembly shown in FIG. 1, the left side elevation view being a mirror image thereof.

FIG. 4 is an exploded side elevation view of the sensor assembly shown in FIG. 1.

FIG. 5 is a perspective view of a fluid inlet shown in FIG. 4 according to one aspect of the invention.

FIG. 6 is a top plan view of the fluid inlet shown in FIG. 5.

FIG. 7 is a side elevation view of the fluid inlet shown in FIG. 5.

FIG. 8 is a bottom view of the fluid inlet shown in FIG. 5.

FIG. 9 is a detailed view, partially in cross-section, of the sensor assembly shown in FIG. 3 as identified by Detail 9 in FIG. 3.

FIGS. 10 through 18 are schematic illustrations of further inlet and housing configurations according to aspects of the invention.

FIG. 19 is a schematic illustration of another inlet and housing configuration where the inlet is remote to the housing according to an aspect of the invention.

FIG. 20 is a schematic elevation view of a sensing device according a further aspect of the invention.

FIG. 21 is a typical plot of a time-dependent concentration of a regulated substance, in this case, THC, after consumption which illustrates a typical presence of the regulated substance immediately after and days after consumption.

### DETAILED DESCRIPTION OF THE INVENTION

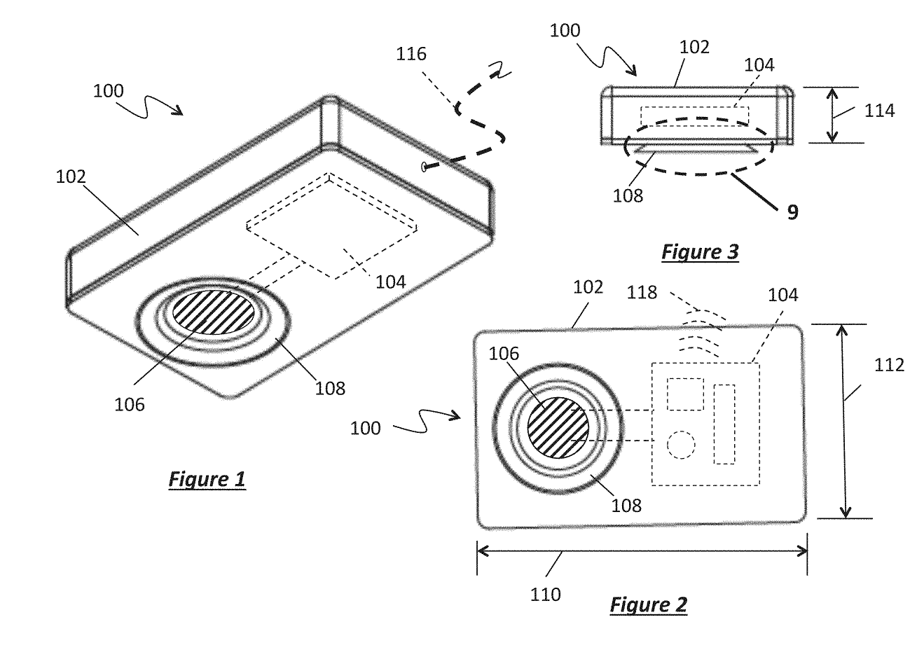

FIG. 1 is a perspective view of a sensor assembly 100 according to one aspect of the invention. Sensor assembly 100 may include a housing 102 containing electronics or electrical devices 104 (shown in phantom), one or more sensors 106 operatively connected to the electronics 104, and/or an inlet 108 that exposes the one or more sensors 106 to a fluid (not shown). FIG. 2 is a bottom view of the sensor assembly 100 shown in FIG. 1. FIG. 3 is a right side elevation view of the sensor assembly 100 shown in FIG. 1, the left side elevation view being a mirror image thereof.

According to aspects of the invention, the fluid to which one or more sensors 106 is exposed may be a liquid (for example, human sweat, blood, or saliva) or a gas (for example, evaporated human sweat). One or more sensors 106 may include some form of sensing surface or sensing element (not shown) that is adapted to vary or generate an electrical signal when exposed to a target, for example, a pre-defined target substance, such as an alcohol, a toxin, or a lipid, among other target substances; or a target condition, such as temperature, humidity, or pH, among other target conditions. Examples of fluids and targets are discussed below. Additional examples of fluids and targets may be found in U.S. Provisional Patent Application 62/340,330, incorporated by reference herein (e.g., see Appendix to U.S. Provisional Patent Application 62/340,330).

As shown in FIGS. 1 through 3, in one aspect of the invention, housing 102 of sensor assembly 100 may be generally rectangular cylindrical in shape, for example, comprising a parallelepiped. In the following discussion, for the sake of simplicity, the rectangular cylindrical shape of housing 102 is assumed. However, it is envisioned that housing 102 may take many physical sizes and shapes while providing substantially the same benefits and advantages over the prior art. For example, housing 102 may be circular cylindrical in shape, elliptical cylindrical in shape, triangular cylindrical in shape, polygonal cylindrical in shape (for example, hexagonal cylindrical or octagonal cylindrical), among other three-dimensional shapes.

The size of housing 102 may vary depending upon the specific requirements of the application to which sensor assembly 100 is used. For example, as shown in FIGS. 2 and 3, housing 102 may have a length 110 ranging from about 0.50 millimeters [mm] to about 250 mm. Some example embodiments may have a length 110 of between about 25 mm and about 100 mm, for example, about 50 mm. Housing 102 may have a width 112 ranging from about 0.50 mm to about 250 mm. Some example embodiments may have a width 112 of between about 10 mm and about 100 mm, for example, about 25 mm. Housing 102 may have a thickness 114 ranging from about 0.25 mm to about 100 mm. Some example embodiments may have a thickness 114 of between about 5 mm and about 50 mm, for example, about 10 mm.

According to aspects of the invention, inlet 108 may be located and shaped to assist in the capture of fluids having the target substance and/or target condition, and, in one aspect, to enhance the detection of the target substance and/or target condition. For example, in one aspect, inlet 108 may be adapted to isolate a fluid within the open space of the inlet when the inlet contacts a surface, for example, the skin of a human subject. In one aspect, the inlet may include an open cavity having a substantially continuous distal perimeter, for example, a perimeter distal from or displaced from the sensor 106. In one aspect, the continuous distal perimeter may contact a surface, for example, the skin of a human subject, and provide a seal between the continuous distal perimeter and the surface whereby little or no ambient fluid (for example, ambient air) may enter the substantially sealed cavity. According to aspects of the invention, this isolation or sealing of the open cavity can enhance the detection of the target substance and/or condition, for example, by minimizing or preventing the displacement and/or dilution of the fluid being captured or isolated by the inlet 108. It is envisioned that by providing a relatively greater concentration of, for example, the target substance in the fluid, for instance, a relatively greater concentration of perspiration vapor molecules per unit volume, a more precise or accurate detection of the target in the fluid can be obtained.

In one aspect, the distal perimeter of the inlet 108 may comprise a flexible or elastomeric material having a flexibly or pliability that can enhance the desired sealing function between the distal perimeter and the surface being contacted.

As also shown in FIGS. 1 through 3, the electronics 104 in sensor assembly 100 may be in communication with an external receiver (not shown), such as, with a local area network or a handheld device, by wire or wirelessly. For example, as shown in FIG. 1, in one aspect, sensor assembly 100 may be wired to an external receiver by one or more wires or cables 116 (shown in phantom). In another aspect, the electronics 104 may include a wireless transmitter and may be in communication with an external receiver via a conventional wireless protocol, as indicated schematically by signal 118 in FIG. 2. Example wireless protocols that may be used include Bluetooth®, Wi-Fi, near-field communication (NFC), or radio, among others.

The electronics 104 in in sensor assembly 100 may include any of the hardware and/or software as disclosed herein. For example, input and output devices, storage devices, and/or processors, and the like.

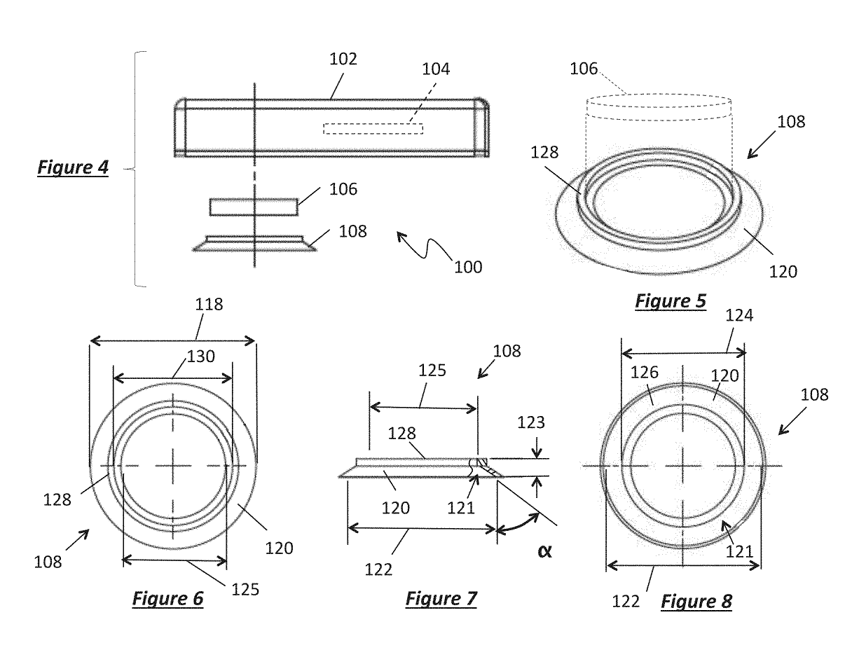

FIG. 4 is an exploded side elevation view of the sensor assembly 100 shown in FIG. 1. As shown in FIG. 4, sensor assembly 100 may include a housing 102 having electronics 104, one or more sensors 106, and an inlet 108. According to aspects of the invention, the one or more sensors 106 employed may comprise any sensor adapted to detect the desired target. Though in the schematic diagram shown in FIG. 4 sensor 106 is shown as generally circular cylindrical in shape, this shape is provided here simply to facilitate disclosure of aspects of the invention. It is envisioned that sensor 106 may comprise any conceivable size and/or shape sensor as dictated by the sensor provider and the dimensions of housing 102 and inlet 108. For example, in one aspect, sensor 106 may be a fuel-cell-type sensor, for example, a fuel-cell-type sensor provided by Dart Sensors Ltd. of Exeter, England, for example, a fuel-cell-type alcohol sensor having model number DS16C-USW, or its equivalent. In another aspect, sensor 106 may be a sensor provided by Hanwei Electronics Co. Ltd., for example, a Hanwei MQ-3 gas sensor disclosed in the Hanwei Gas Sensor Modules spec sheet version 2.2, dated Aug. 2, 2010, provided by Parallax Inc. of Rocklin, California (which is incorporated by reference herein in its entirety), or its equivalent.

FIG. 5 is a perspective view of one fluid inlet 108 according to one aspect of the invention. FIG. 6 is a top plan view of the fluid inlet 108 shown in FIG. 5. FIG. 7 is a side elevation view of the fluid inlet 108 shown in FIG. 5 and FIG. 8 is a bottom view of the fluid inlet 108 shown in FIG. 5.

As shown in FIGS. 5 through 8, according to one aspect of the invention, fluid inlet 108 may comprise a conical, or frusto-conical, ring or annular structure 120 having an outside diameter 118, an upper internal diameter 124, and a larger, lower diameter 122. The upper internal diameter 124 and the lower internal diameter 122 may define an internal cavity 121. According to one aspect of the invention, conical ring 120 may have a continuous sidewall or inner surface 126, which may be smooth and continuous, between the upper diameter 124 and lower diameter 122. According to aspects of the invention, this continuous sidewall or inner surface 126, for example, a conical, smooth, and continuous surface, may function to capture fluid so that target substances and/or condition of the fluid can be detected by sensor 106, for example, more readily and more accurately detected.

As shown in FIG. 7, in one aspect, the smooth, continuous surface 126 of ring 120 may define an angle α with the direction of the axis of ring 120, for example, an angle of inclination or an angle of taper. The angle α may range from about 5 to about 85 degrees. In some embodiments, the angle α may range between about 40 degrees and about 60 degrees, for example, about 45 degrees. In one aspect, the sidewall or inner surface 126 of ring 120 may not be tapered, but may comprise a substantially non-tapered, for example, vertical, internal sidewall of ring 120.

In another aspect, as shown in FIGS. 5 through 8, inlet 108 may include a ring or annular structure 128 mounted to or integral with conical ring 120. In one aspect, ring 128, or an upper ring, may provide an interface between ring 120 and housing 102. In another aspect, for example, as shown in FIG. 5, ring 128 may provide a receptacle or recess for receiving the one or more sensors 106 (shown in phantom). For example, as shown in FIG. 6, ring 128 may have an internal recess dimension (that is, diameter or width) 130 adapted to receive the one or more sensors 106 and an internal opening dimension (that is, diameter or width) 125 sized to expose the one or more sensors 106 to the cavity 121 within conical ring 120. Though shown as a circular ring in FIGS. 5 through 8, it is envisioned that the structure described as ring 128 may not be circular, but may also be elliptical or polygonal, for example, square, and provide the desired function of retaining one or more sensors 106 in inlet 108 while exposing the one or more sensors 106 to the cavity 121 within ring 120.

The size and dimensions of inlet 108 shown in FIGS. 4 through 8, and of any inlet disclosed herein, may vary as a function of application and use of aspects of the invention. For example, the outside width or diameter 118 of inlet 108 may range from about 5 mm to about 300 mm (about 1 foot). In some embodiments, outside diameter 118 may range from about 10 mm to about 50 mm, for example, about 25 mm. The thickness 123 of inlet 108 may range from about 2 mm to about 50 mm (about 2 inches). In some embodiments, thickness 123 may range from about 5 mm to about 20 mm, for example, about 15 mm.

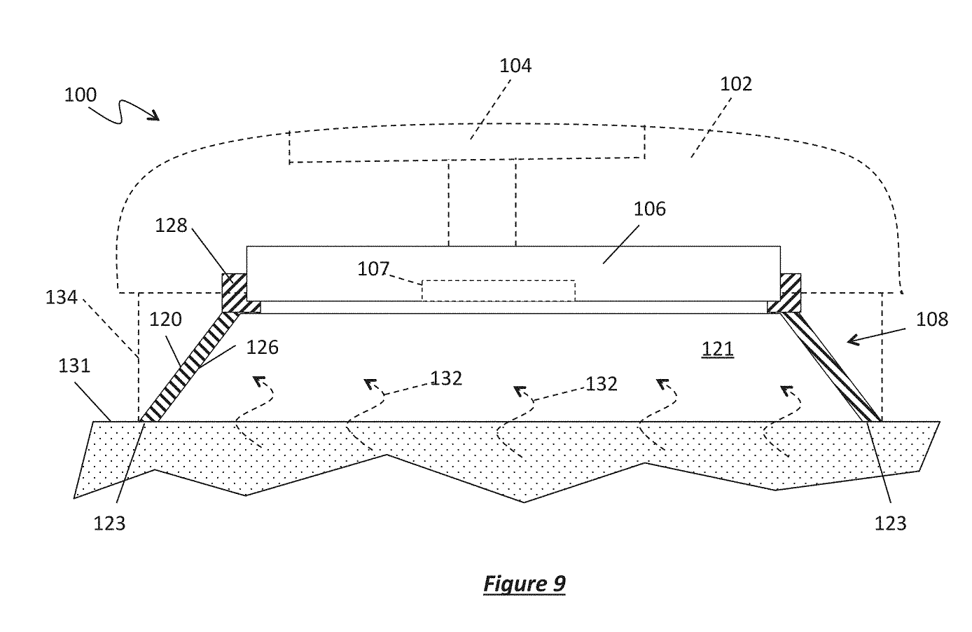

FIG. 9 is a detailed view, partially in cross-section, of the sensor assembly 100 shown in FIG. 3 as identified by Detail 9 in FIG. 3. FIG. 9 also illustrates the presence of a representative surface 131 upon which sensor assembly 100 may be applied or contacted according to aspects of the invention. For example, surface 131 may be the surface of the skin of a subject, for instance, a human or animal subject, from which a fluid 132 (shown in phantom) having a target to be detected emanates, for example, perspiration or “sweat.”

As shown in FIG. 9, according to aspects of the invention, inlet 108 mounted to housing 102 may contact surface 131. Housing 102 may contain electronics 104, as described herein. Inlet 108 may include conical ring 120 and upper ring 128, which may retain one or more sensors 106 having one or more sensing elements 107 (shown in phantom). According to aspects of the invention, conical ring 120 may include a distal perimeter or distal bearing surface 123 that is adapted to contact surface 131, and, when in contact, provide a substantially enclosed cavity 121 within inlet 108. According to an aspect of the invention, the internal tapered surface or sidewall 126 of ring 120 may function to enlarge the area of surface 131 captured by inlet 108 compared to the relatively smaller area of the one or more sensors 106, for example, the relatively smaller area of the sensing element 107 of the one or more sensors 106. Accordingly, it is envisioned that this encompassing of a relatively larger area by the distal perimeter 123 of inlet 108 may enhance and isolate, for example, from undesirable dilution by the ambient environment and, for example, any ambient air currents, the capture of fluid 132. This capture and isolation of fluid 132, for example, perspiration, can enhance the detection of target substances and/or conditions within fluid 132 by the one more sensors 106, and thus provide a more accurate and reliable detection of target substances and/or conditions.

For example, in one aspect, the area of the surface 131 that may be captured by distal perimeter 121 may be at least 10% greater than the surface area of the one or more sensors 106, or, more specifically, 10% greater than the surface area of the sensing elements 107 of sensors 106. In another aspect, the area of the surface 131 that may be captured by distal perimeter 123 may be at least 20% greater or at least 30% greater than the area of the one or more sensors 106 (or the area of the sensing element 107). In a further aspect, the area of the surface 130 that may be captured by distal perimeter 123 may be at least 40% greater or at least 50% greater than the area of the one or more sensors 106 (or the area of the sensing element 107).

FIG. 9 also illustrates that the shape of inlet 108 may not be limited to the external shape of the conical ring 120. Specifically, according to one aspect of the invention, the contact and capture of the surface 131 by distal perimeter 123 and internal sidewall or surface 126 may be provided by a broad range of external geometries of inlet 108. For example, as shown in FIG. 9, inlet 108 may comprise an annular or ring-like structure with an undefined external shape or dimension 134 (shown in phantom) while still providing the desired contact and isolation function of distal perimeter 123 and internal sidewall 126. In other words, according to one aspect of the invention, it is the internal structure of inlet 108, that is, the dimension of distal perimeter 123 and the shape of internal surface 126 that can be sufficient to provide the desired capture and isolation of fluid 132, where the external dimensions of inlet 108 may not affect these desired functions.

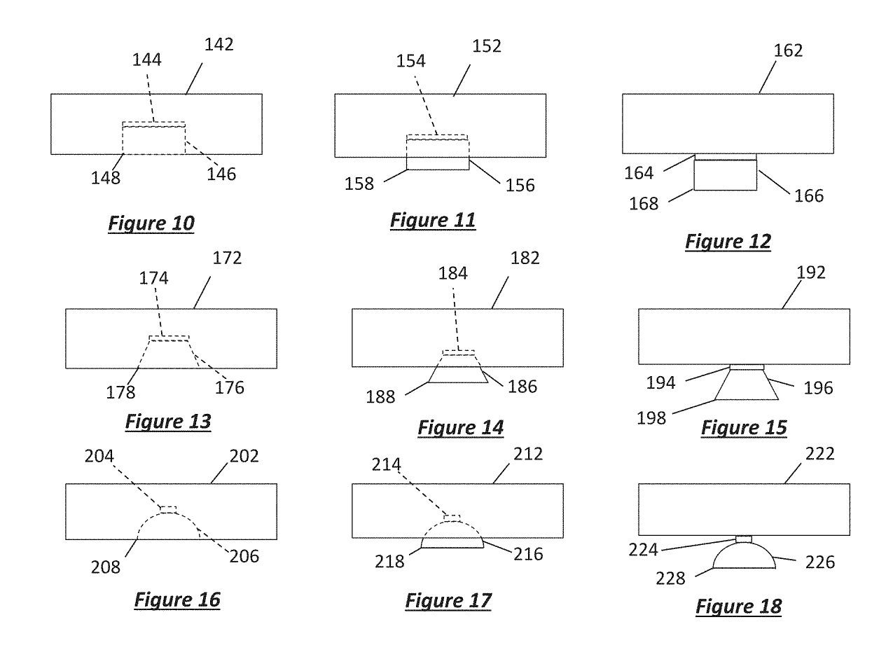

FIGS. 10 through 18 are schematic illustrations of other envisioned inlet and housing configurations, the housings having appropriate electronics (not shown), according to aspects of the invention. According to aspects of the invention, the positioning and shape of inlet 108 with respect to housing 102, for example, within, outside, or remote of housing 102, may vary without detracting from the effectiveness of aspects of the invention from providing the desired functions. For example, as shown in FIG. 10, an inlet 146 having a distal perimeter 148 and one or more sensors 144 may be positioned within a housing 142, and the distal perimeter 148 of inlet 146 may provide the desired isolation of the surface being contacted. As also illustrated in FIG. 10, according to one aspect, inlet 146 may comprise a circular cylindrical, elliptical cylindrical, or polygonal cylindrical shape having generally non-tapered sidewalls.

As shown in FIG. 11, a circular cylindrical, elliptical cylindrical, or polygonal cylindrical inlet 156 having a distal perimeter 158 and one or more sensors 154 may be positioned partially within a housing 152, and the distal perimeter 158 of inlet 156 may provide the desired isolation of the surface being contacted.

As shown in FIG. 12, a circular cylindrical, elliptical cylindrical, or polygonal cylindrical inlet 166 having a distal perimeter 168 and one or more sensors 164 may be positioned externally of a housing 162, and the distal perimeter 168 of inlet 166 may provide the desired isolation of the surface being contacted.

As shown in FIG. 13, a conical inlet 176, similar to inlet 108 disclosed herein, having a distal perimeter 178 and one or more sensors 174 may be positioned within a housing 172, and the distal perimeter 178 of conical inlet 176 may provide the desired isolation of the surface being contacted. As also illustrated in FIG. 13, according to one aspect, inlet 176 may comprise a frusto-conical circular cylindrical, frusto-conical elliptical cylindrical, or a frusto-conical polygonal cylindrical shape having generally tapered sidewalls, for example, having an angle of taper a as shown in FIG. 7.

As shown in FIG. 14, a conical inlet 186 having a distal perimeter 188 and one or more sensors 184 may be positioned partially within a housing 182, and the distal perimeter 188 of conical inlet 186 may provide the desired isolation of the surface being contacted.

As shown in FIG. 15, a conical inlet 196 having a distal perimeter 198 and one or more sensors 194 may be positioned externally of a housing 192, and the distal perimeter 198 of inlet 196 may provide the desired isolation of the surface being contacted.

As shown in FIG. 16, a hemispherical inlet 206 having a distal perimeter 208 and one or more sensors 204 may be positioned within a housing 202, and the distal perimeter 208 of hemispherical inlet 206 may provide the desired isolation of the surface being contacted.

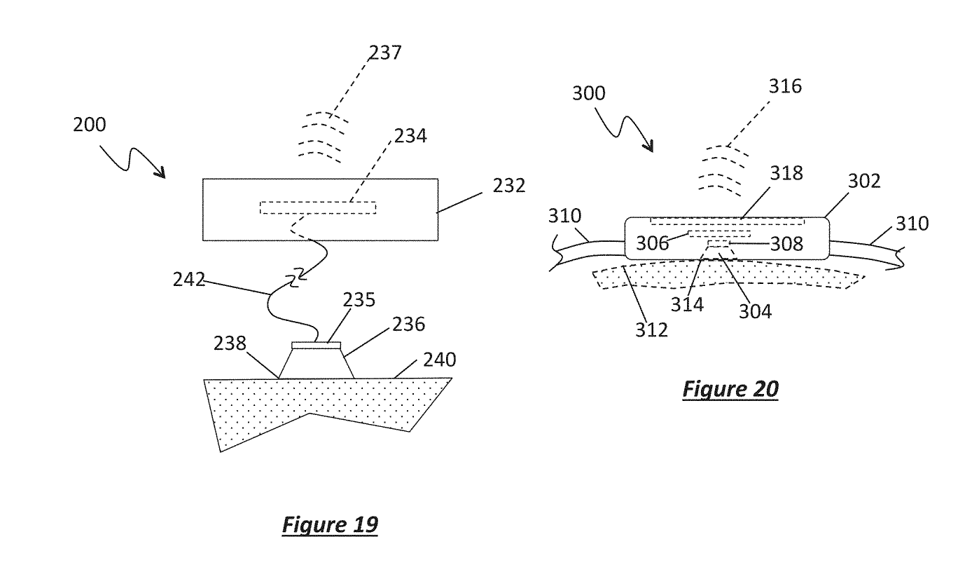

As shown in FIG. 17, a hemispherical inlet 216 having a distal perimeter 218 and one or more sensors 214 may be positioned partially within a housing 212, and the distal perimeter 218 of hemispherical inlet 216 may provide the desired isolation of the surface being contacted.

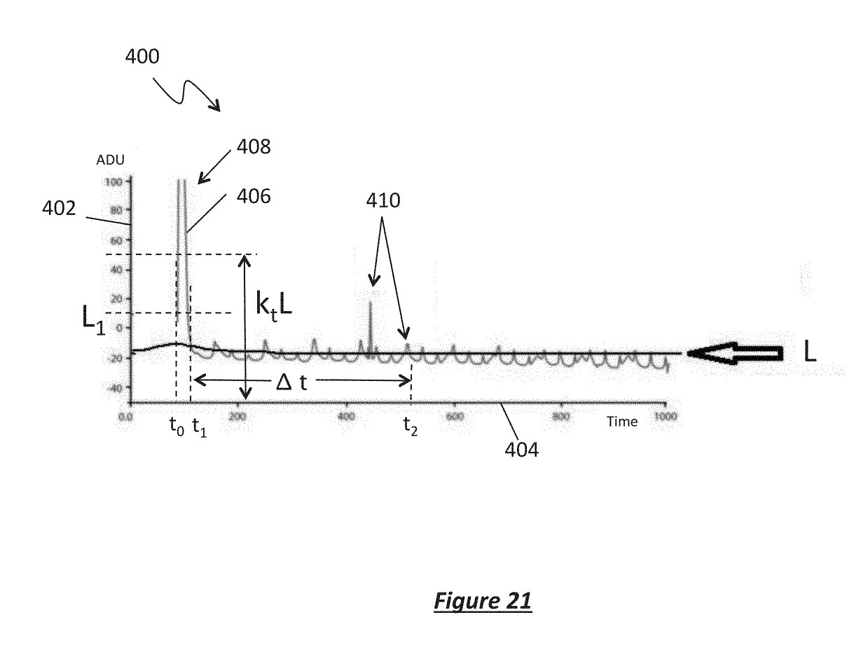

As shown in FIG. 18, a hemispherical inlet 226 having a distal perimeter 228 and one or more sensors 224 may be positioned externally of a housing 222, and the distal perimeter 228 of inlet 226 may provide the desired isolation of the surface being contacted.

FIG. 19 is a schematic illustration of another inlet and housing system 200 where the inlet is remote to the housing according to an aspect of the invention. As shown in FIG. 19, system 200 may include a housing 232, for example, a housing similar to and having the attributes of any one of the housings (such as, housing 102) disclosed herein, and an inlet or inlet module 236, for example, an inlet similar to and having the attributes of any one of the inlets (such as, inlet 108) disclosed herein. Housing 232 may include electronics 234 similar to the electronics disclosed herein.

In the aspect of the invention shown in FIG. 19, inlet 236 may comprise a separate, remote module having one or more sensors 235, as disclosed herein. As is typical with aspects of the invention disclosed herein, inlet 236 may include a distal perimeter 238, and inlet 236 may be mounted to or contact a surface 240. Surface 240 may be any surface, for example, the skin of a subject, wherein inlet 236 may isolate and expose one or more sensors 235 to a fluid, for example, and perspiration from surface 240. The electrical signals corresponding to the substance or condition detected by the one or more sensors 235 may then be communicated to electronics 234 in housing 232 via one or more wires 242 or wirelessly. Also, signals corresponding to the substance or condition detected may be transmitted by electronics 234 to an external receiver (not shown), for example, by wire or wireless, as indicated by wireless signal 237.

FIG. 20 is a schematic elevation view of a sensing device 300 according to a further aspect of the invention. As shown in FIG. 20, device 300 may include a housing 302, for example, a housing similar to and having the attributes of any one of the housings (such as, housing 102) disclosed herein, and an inlet 304, for example, an inlet similar to and having the attributes of any one of the inlets (such as, inlet 108) disclosed herein. Housing 304 may include electronics 306 similar to the electronics disclosed herein, and one or more sensors 308, as disclosed herein.

In the aspect of the invention shown in FIG. 20, device 300 may include some form of restraining device, mechanism, or strap 310 adapted to retain device 300 on a surface 312. Only representative portions of restraining device 310 are shown in FIG. 310; however, it is envisioned that restraining device 310 can be similar to other band-type devices known in the art. For example, restraining device 310 may extend about a body or a structure to which device 300 is mounted and engage a coupling device or clasp (not shown). In one aspect, the surface 312 may be any surface of a human body or of an animal body. For example, in one aspect, device 300 may comprise a “wrist watch”-type device, where restraining device 310 comprises one or more bands or straps, for instance, a watchband, and surface 312 comprises a wrist of a wearer. In other aspects of the invention, restraining device 310 may comprise an armband or a leg band where surface 312 may be the surface of the skin of an arm or a leg of a wearer. In another aspect, restraining device 310 may be adapted to mount device 300 about the abdomen, chest, or back of the wearer. In one aspect, the restraining device may comprise a “head band” where the surface 312 may comprise a surface of the head of a wearer.

As is typical with aspects of the invention disclosed herein, inlet 304 may include a distal perimeter 314 that contacts surface 312. Surface 312 may be any surface, for example, the skin of a subject, wherein inlet 304 may isolate and expose one or more sensors 308 to a fluid, for example, perspiration from surface 312. The electrical signals corresponding to the substance or condition detected by the one or more sensors 308 may then be communicated to electronics 306 in housing 232 via one or more wires or wirelessly. In this aspect, electrical signals corresponding to the substance or condition detected by one or more sensors 308 may be transmitted by electronics 306 to an external receiver (not shown), for example, by wire or wirelessly, as indicated by wireless signal 316.

In one aspect, the electronics 306 may transmit a signal to a display 318 mounted in housing 302, for example, for viewing by the wearer of device 300, for example, for substantially immediate display and reviewing by the wearer. Display 318 may be a light-emitting diode (LED) type display or a liquid crystal display (LCD), among other types of displays, as known in the art.

As disclosed herein, aspects of the invention may employ a broad range of sensors adapted to detect a broad range of fluids, substances within the fluids, and conditions of the fluids. For example, the one or more sensors disclosed herein, for example, sensor 106 shown in FIG. 2 or sensor 235 shown in FIG. 19, may be adapted to detect one or more conditions of the fluid and/or any components of the fluid, for example, any analyte present in a gas or liquid. The substances that may be detected by the sensors disclosed herein may include, but are not limited to, alcohols, ions, metabolites, proteins, salts, fats, sugars, lipids, enzymes, amino acids, nucleotides, genes, antioxidants, organic acids, drugs, narcotics, pharmaceuticals, chemicals (such as, tetrahydrocannabinol (THC) or THC metabolites, or anti-bodies to THC), vitamins, melatonin, electrolytes, carbon dioxide, carbon monoxide, among others.

Also, the one or more sensors disclosed herein may be adapted to detect one or more of the following conditions of the fluid: temperature, humidity, conductivity, resistivity, pH, alkalinity, acidity, and viscosity, among others. The detection of any one or more of these conditions may be practiced alone, or may be practiced while detecting one or more substances.

The material of the housings, for example, housing 102, and the material of the inlets, for example, inlet 108, may vary broadly depending upon the application of use of aspects of the invention and/or the environment of use. For example, the housings and the inlets disclosed herein may be metallic or non-metallic. For example, in one aspect, the housings and the inlets may be made from a metal, for example, an aluminum, a steel, a stainless steel, or a titanium, among other metals and metal alloys. In another aspect, the housings and the inlets may be made from a plastic, an elastomer, or a wood. In one aspect, the housings and the inlets may be made from one or more plastics, for example, a polyamide (PA), for example, nylon; a polyethylene (PE), both high-density polyethylene (HDPE) and low-density polyethylene (LDPE); a polyethylene terephthalate (PET); a polypropylene (PP); a polyester (PE); a polytetrafluoroethylene (PTFE); a polystyrene (PS); an acrylonitrile butadiene styrene (ABS); a polycarbonate (PC); or a polyvinylchloride (PVC); among other plastics. In another aspect, the housings and the inlets may be made from one or more elastomers, for example, a natural polymer, such as, polyisoprene rubber, or a synthetic polymer, such as, a neoprene, a thermoplastic elastomer, a thermoplastic rubber, and a polyvinyl chloride, an ethylene propylene diene monomer (EPDM) rubber, or a polydimethylsiloxane (PDMS), and the like.

In one aspect, at least the housings, for example, housing 102, may comprise an elastomeric or pliant or flexible material to enhance the flexibility and compliancy of the housing with a surface upon which the housing is mounted. For example, when the housing is used as a “patch” mounted upon an uneven or flexible surface, for example, the skin of a subject, a flexible or compliant elastomeric material may better conform to the surface and be more comfortable to the subject.

In one aspect, at least the distal perimeter of the inlet, for example, distal perimeter 123 of inlet 108 may comprise an elastomeric or pliant or flexible material to enhance the contact and/or sealing effect of the distal perimeter upon the mating surface, for example, the skin of a subject. In one aspect, the inlet ring, for example, ring 120 of inlet 108, may be made of an elastomeric or pliant or flexible material to enhance the contact and/or sealing effect of the distal perimeter upon the mating surface.

In one aspect, the methods, systems, and devices disclosed herein may be adapted to provide for the detection of restricted substances, for example, narcotics, in a fluid, for example, in a gas or in a liquid. In one aspect, the restricted substance may be a cannabinoid from the ingestion of marijuana, specifically, a tetrahydrocannabinol (THC), for example, delta9-tetrahydrocannabinol, or a THC metabolite. In one aspect, the methods and systems disclosed herein may provide enhanced detection of subject impairment when the subject has ingested a restricted substance, such as, a THC.

As known in the art, the active constituent of cannabis, THC and/or THC metabolites, can linger in the human body long after a person has smoked or otherwise ingested a THC-containing substance. These constituents can be ingested, for example, by inhaling the smoke of marijuana, hashish, bang, or ganja; by consuming marijuana-containing food; or by ingesting a tablet containing THC. It is recognized in the art that THC or THC metabolites or THC anti-bodies may be detectable in bodily fluids days, and even weeks after ingestion (Huestis, et al., 2007), and long after any deleterious impairments of psycho-motor skills and/or cognition have occurred. That is, THC or THC metabolites or THC anti-bodies can be detectable in bodily fluids while the user is substantially “sober” and “unimpaired.”

As also known in the art, “THC metabolites,” that is, substances that may be produced when the human body metabolizes THC, are numerous, and some of these metabolites may be psycho-active and some non-psycho-active. For example, it is known that the primary psycho-active metabolite of THC is 11-hydroxy-delta9-tetrahydrocannabinol (11-OH-THC), and the primary non-psycho-active metabolite is 11-nor-9-carboxy-delta9-tetrahydrocannabinol (THC-COOH) [Sharma, et al., 2012].

It is also recognized in the art that one reason for the residual presence of THC or THC metabolites in a bodily fluid is that, unlike alcohol and other narcotics (such as, “meth,” or opiates), THC and/or THC metabolites are typically fat-soluble. That is, after ingestion, THC and/or THC metabolites may be absorbed and maintained in fat tissue (that is, adipose tissue) long after ingestion has ceased. Subsequently, these THC and/or THC metabolites, for example, non-psycho-active THC metabolites, can be released back into the body, for example, into the blood stream, when the fat tissue containing the THC and/or THC metabolites is metabolized, that is, “burned,” by the body to yield energy. It is understood that some THC metabolites may be present and detectable up to four weeks after ingestion (depending, for example, upon usage habits). Hence, unwitting users of restricted drugs like marijuana may be subject to statutory liabilities, social or professional stigmas, or professional liabilities long after the consumption, for example, legal consumption, of THC-containing substances has occurred.

Recognizing this potential time-difference between the time of consumption and the time that THC metabolites may be detectable, but with little or no impairment, aspects of the present invention were conceived and developed.

First, it was recognized that the presence of THC and THC metabolites, or other regulated substances, in bodily fluids, for example, in the blood stream or in sweat, alone is unlikely to be an indicative of an individual's “impairment.” As known in the art, impairment due to the ingestion of a psycho-active substance can be manifested by, for example, a person's decrease in psycho-motor skills, decrease in cognition, and/or decrease in ability to safety drive a vehicle. As noted above, THC metabolites may be detectable in the blood stream days after ingestion when an individual is not impaired.

Second, it is recognized that other biomarkers have been identified that reflect, or are more consistent with, an individual's relative impairment. In response, systems and methods disclosed herein may consider both the presence of a regulated substance in a bodily fluid and the presence of a biomarker and/or a heath characteristic associated with an individual's impairment and, then, provide an indication of the individual's impairment due to the presence of the regulated substance in a bodily fluid.

According to one aspect of the invention, a method of monitoring regulated material levels, for example, a THC or THC metabolite levels, in a subject is provided. This method may comprise or consist of a) detecting a regulated material concentration, such as, a THC or a THC metabolite concentration, in a first fluid of the subject, for example, a bodily fluid; b) detecting a concentration of a biomarker reflective of impairment of the subject in a second fluid of the subject, for example, a bodily fluid; c) comparing the regulated material concentration in the first fluid with a predetermined regulated material concentration limit; d) comparing the concentration of the biomarker with a predetermined biomarker concentration limit; and/or e) when the detected regulated material concentration varies from, for example, exceeds, the predetermined regulated material concentration limit and when the concentration of the biomarker varies form, for example, exceeds, the predetermined biomarker concentration limit, identifying the subject as impaired. According to some aspects, “impaired” may mean a relative decrease in psycho-motor skills, a relative decrease in cognition, and/or a relative decrease in ability to safety drive a vehicle, for example, a car, a boat, or a plane.

In one aspect of the invention, detecting may comprise monitoring, for example, substantially continuously monitoring the regulated material concentration and/or the concentration of a biomarker in the fluid, for example, with an appropriate sensor. In another aspect, detecting may comprise intermittently monitoring, for example, with an appropriate sensor.

In one aspect of this method, the regulated material may be a THC or a THC metabolite. In another aspect, the first fluid and the second fluid may be the same fluid, for example, a blood sample of the subject. In one aspect, the first fluid and the second fluid may be a bodily fluid, for example, blood, sweat, tears, saliva, breath from nose or mouth, or a combination thereof.

Aspects of this method may utilize one or more of the sensors, sensor housings, and/or sensor inlets as disclosed herein.

In one aspect, the predetermined regulated material concentration limit may be a concentration of THC (delta-9-tetrahydrocannabinol), for example, a concentration of about 2 nanograms of THC per milliliter of liquid [nag/ml] to about 12 nag/ml, for instance, 5 nag/ml.

In one aspect, the biomarker may be a neurotransmitter, such as, serotonin; an enzyme, such as, creatine kinase-myocardial b fraction (CK-MB) or amylase (for example, in saliva) [Seugnet, et al., 2006]; a lactic acid (Mishra, 2015); blood sugar; blood urea; creatinine [Nayak, et al., 2012]; a melatonin; or a peptide, such as, a small-molecular-weight protein or peptide (for example, in saliva) [Michael, et al., 2013 an 2011]; among other biomarkers.

In one aspect of the invention, instead of or in addition to detecting a biomarker, the method may comprise detecting a health or behavior characteristic of the subject under examination. For example, in one aspect, at least one of an actigraphy, an electroencephalography (EEG), a multiple sleep latency test (MSLT), a reaction time, a pupillography, a metabolic rate, a body temperature, a heart rate, and a heart-rate variability [Michael, et al., 2013], among other health characteristics, may be examined or detected and then compared to one or more corresponding predetermined health characteristic criteria. In one aspect, evidence of driving performance, for example, recent or simultaneous evidence of “weaving on a road,” may be detected. In another aspect, a fluid component, for example, a blood, sweat, tears, or saliva component, such as, a metabolite, which can be correlated with lack of focusing, may be detected.

Accordingly, in one aspect of the invention, the above steps b) and d) above may be supplemented or replaced by f) detecting a health characteristic of the subject; and g) comparing the detected health characteristic with a predetermined health characteristic criterion. In one aspect, the method may further comprise h) when the detected regulated material concentration varies, from, for example, exceeds, the predetermined regulated material concentration limit and when the health characteristic varies from, for example, exceeds, the predetermined health characteristic criterion, identifying the subject as impaired.

According to another aspect of the invention, a system for monitoring restricted substances, such as, a THC or a THC metabolite, in a subject is provided. This system may comprise or consist of a first sensor adapted to detect a regulated material concentration, such as, a THC and/or a THC metabolite concentration, in a first fluid of the subject, for example, a bodily fluid; a second sensor adapted to detect a concentration of a biomarker reflective of impairment of the subject in a second fluid of the subject, for example, a bodily fluid; and a processor adapted to compare the regulated material concentration in the first fluid with a predetermined regulated material concentration limit, adapted to compare the concentration of the biomarker with a predetermined biomarker concentration limit, and, when the detected regulated material concentration varies from, for example, exceeds, the predetermined regulated material concentration limit and when the concentration of the biomarker varies from, for example, exceeds, the predetermined biomarker concentration limit, identify the subject as impaired.

In one aspect of this system, the regulated material may be a THC or a THC metabolite. In another aspect, the first fluid and the second fluid may be the same fluid, for example, a blood sample of the subject. In one aspect, the first fluid and the second fluid may be a bodily fluid, for example, blood, sweat, saliva, tears, breath from nose or mouth, or a combination thereof. In one aspect, the processor may be a one or more processor.

Aspects of this system may utilize one or more of the sensors, sensor housings, and/or sensor inlets disclosed herein.

In one aspect, the above system, the second sensor may be supplemented or replaced by a sensor adapted to detect a health characteristic of the subject, for example, one or more of the health characteristics referenced above, and the processor may be adapted to compare the detected health characteristic with a predetermined health characteristic criterion. In one aspect, the processor may further be adapted to, when the detected regulated material concentration exceeds the predetermined regulated material concentration limit and when the health characteristic varies from the predetermined health characteristic criterion, identify the subject as impaired.

In another aspect of the invention, methods and systems are provided which may recognize that the presence of a regulated material in a bodily fluid, such as, blood, sweat, tears, and/or saliva, may rise abruptly soon after consumption of the regulated material, and then decrease; but, as noted above, the presence of the regulated material may still be detectable in the bodily fluid well after the time of consumption. Again, as noted, it is understood that a fat-soluble regulated material, such as, THC, may continue to be released into, for example, the blood stream, and be detectable days, if not weeks, after consumption of the regulated material.

FIG. 21 is an example plot 400 of a time-dependent concentration of a regulated substance, in this case, THC, after consumption which illustrates a presence of the regulated substance immediately after and hours or days after consumption. In FIG. 21, the y-axis, or ordinate, 402 represents the concentration of THC as indicated by an arbitrary detection unit (ADU), since many types of detection units are possible in the art; the x-axis, or abscissa, 404 represents duration or time, in arbitrary time units. The curve 406 represents the time variation of THC in arbitrary detection units. Plot 400 also illustrates an example legal limit, L, for presence of THC in the blood stream of the subject, again, in arbitrary detection units.

As shown in FIG. 21, soon after ingestion or consumption of the regulated material, for example, at a time t₀ soon after smoking a marijuana cigarette, the concentration of THC in the blood may rise rapidly, as indicated by peak 408 in curve 406. Clearly, as shown in FIG. 21, the concentration 406 in the blood during peak 408 may be greater, for example, much greater, than a legal limit L. Accordingly, with such a relatively high concentration in the blood, depending upon the usage habits, gender, size, and/or body mass index of the subject, among other things, it may be likely that during and shortly after the time of peak 408, the subject may be impaired, as discussed herein.

However, as also shown in FIG. 21, the concentration 406 may relatively quickly diminish, for example, at time t₁, and, though not always lower than the legal limit L, the concentration 406 may subsequently be dramatically lower than the concentrations indicated by peak 408. In addition, FIG. 21 illustrates smaller peaks of the concentration curve 406, for example, at peaks 410. It is understood that these peaks 410, for example, may represent short-term THC concentration increases, for example, for 20 to 30 minutes, due to the metabolism of adipose tissue (that is, fat) containing fat-soluble THC. Accordingly, as investigations in the art have indicated, the concentration of THC in the blood stream may be detectable days or even weeks after consumption has ended due to the metabolism of THC-containing fat, while the subject is typically not impaired, for example, even if the concentration 406 is at or above the legal limit L. Aspects of the present invention were conceived and developed to address this time-dependency of THC concentration, and other regulated materials, and the impact upon these concentrations of the timing of the metabolism of THC-containing fat.

In one embodiment of the invention, a method of detecting the sobriety or impairment of a subject due to the consumption of a regulated material, such as, THC, is provided. The method may comprise or consist of a) monitoring a regulated material concentration, such as, a THC or a THC metabolite or THC anti-body concentration, in a fluid of the subject, for example, a bodily fluid; b) comparing the monitored regulated material concentration in the fluid with a predetermined regulated material concentration limit, L; c) when the monitored regulated material concentration exceeds the predetermined concentration limit, L, by a predetermined amount, kₜL, where kₜ is a threshold constant, for example, an absolute or relative variation from the predominated concentration limit, L, defining a start time, t₀; d) after defining the start time, t₀, continuing to monitor the regulated material concentration in the fluid of the subject; e) when the monitored regulated material concentration decreases below a predetermined concentration limit L₁, defining a stop time, t₁, and f), for any time after the stop time, t₁, designating the subject “unimpaired.” In one aspect, the monitoring may comprise substantially continuously monitoring. In another aspect, monitoring may comprise intermittently monitoring. The regulated material may be any one of the regulated materials disclosed herein.

In one aspect, in order to minimize or prevent the premature identification of a subject being unimpaired, the method may comprise defining a later time, t₂, after t₁, where t₂ comprises an additional time, Δt (“delta T”) after stop time, t₁. In one aspect, At may be at least 1 hour, or at least 6 hours, or at least 12 hours, or at least 1 day. Accordingly, in one aspect, step f) of the method may comprise defining a time after t₂, where t₂=t₁+Δt, and only after the stop time, t₂, designating the subject “unimpaired.”

In one aspect, the threshold constant, kₜ, may be greater than 1.0, for example, at least about 1.10 to about 1.30. In one aspect, the threshold constant, kₜ, may be at least 1.50, for example, at least 2.0. In one aspect, kₜ may be 1.0, where time t₀ is defined as the time when the detected concentration reaches kₜ L=L, the legal limit. In other aspects, kₜ may be less than 1.0, for example, between 0 and 1.0. In one aspect, L₁ may be equal to kₜL. In one aspect, L₁ may be equal to L, the legal limit. In one aspect, kₜ may be a percentage of L.

In one aspect, kₜ may be time-dependent, that is, a function of time, kₜ (t). For example, in one aspect, kₜ (t) may vary between time t₀ and time t₂, or between time t₀ and time tₙ, where time tₙ is greater than time t₂. In another aspect, kₜ(t) may be substantially constant for a first time period, tₖ, and then vary over a second time period, for example, decrease. The decrease in the value of kₜ(t) after time tₖ (and the corresponding decrease of the threshold defined by the product kₜ(t)L) may be understood to mimic what is believed to be the natural decay of, for example, THC concentration in the body or blood stream over time. For instance, the value of kₜ(t) may decease after a time tₖ where the concentration threshold may substantially approach zero or disappear with time.

In one aspect, the variation in the value of kₜ(t) may decrease linearly [kₜ(t)=f(t)], quadratically [kₜ(t)=f(t²)], cubically [kₜ(t)=f(t³)], or exponentially [kₜ(t)=f(e^{t})], among other functions of time. For example, in one aspect, kₜ(t) may be constant and then decrease linearly in compliance with Equation 1 and Equation 2,

kₜ(t)=k₀ (for t≦tₖ) Equation 1

kₜ(t)=k₀−mt (for t>tₖ) Equation 2

where k₀ is an initial constant value of kₜ(t) between time t₀ and time tₖ; tₖis the time of the beginning of the variation of kₜ(t) from k₀; and m is the rate of change, (in this case, decrease, or “slope”) of kₜ(t) at times greater than time tₖ.

In another aspect, kₜ(t) may be determined experimentally, for example, from THC variation with time determined from the testing of subjects, for example, the testing the THC concentration of the blood or perspiration of a population of subjects, as known in the art. In one aspect, an appropriate function kₜ(t) may be determined, for example, by “curve fitting” the time-dependent data so obtained. In other aspects, the variation of the value of kₜ(t) may be determined via a database or a “look up table,” for example, based upon data obtained from the testing of subjects. Other methods of establishing the time dependence of the function kₜ(t) will be conceivable by those of skill in the art.

Aspects of these methods may utilize one or more of the sensors, sensor housings, and/or sensor inlets disclosed herein.

In another embodiment of the invention, a system for detecting the sobriety or impairment of a subject due the consumption of a regulated material, such as, THC, is provided. The system may comprise or consist of a sensor adapted to detect a regulated material concentration, such as, a THC or a THC metabolite or THC anti-body concentration, in a fluid of the subject, for example, a bodily fluid; a processor configured to receive an electrical signal from the sensor corresponding with the detected regulated material concentration in the fluid and compare the regulated fluid concentration with a predetermined regulated material concentration limit, L; and, when the monitored regulated material concentration exceeds the predetermined concentration limit, L, by a predetermined amount, kₜL, the processor is configured to define a start time, t₀; and, after defining the start time, t₀, the processor is configured to continue to detect the regulated material concentration in the fluid of the subject; and, when the detected regulated material concentration decreases below a predetermined concentration limit L₁, the processor is configured to define a stop time, t₁, and, for any time after the stop time, t₁, the processor is configured to designate the subject “unimpaired.” In one aspect, the processor may be one or more processors and the sensor may be one or more sensors. The regulated material may be any one of the regulated materials disclosed herein.

In one aspect, the sensor may be configured to substantially continuously detect a regulated material concentration. In another aspect, the sensor may be configured to intermittently detect a regulated material concentration.

In one aspect, in order to minimize or prevent the premature identification of a subject being unimpaired, the processor of the system may be configured to define a later time, t₂, after t₁, where t₂ comprises an additional time, Δt (“delta T”) after stop time, t₁. In one aspect, Δt may be at least 1 hour, or at least 6 hours, or at least 12 hours, or at least 1 day. Accordingly, in one aspect, the processor may be configured to define a time after t₂, where t₂=t₁+Δt, and only after the stop time, t₂, the processor may designate the subject “unimpaired.”

In one aspect, the threshold constant, kₜ, may be greater than 0, for example, at least 0.10 to about 0.30. In one aspect, the threshold constant, kₜ, may be at least 0.50, for example, at least 1.0. In one aspect, L₁ may be equal to kₜL. In one aspect, L₁ may be equal to L, the legal limit. In one aspect, kₜ may be a percentage of L.

In one aspect, one or more of the sensors, sensor housings, and/or sensor inlets disclosed herein may be used to provide a wearable lab-on-a-patch for real-time monitoring of blood alcohol concentration (BAC) sensing, THC sensing, and/or other target sensing. This biosensor may securely communicate with a smartphone application via a mobile health IT cloud system. Moreover, in one embodiment, the addition of a humidity sensor and a temperature sensor (in addition to the disclosed electrochemical sensor) may enhance calibration of the disclosed process, thereby giving more specific and sensitive data; that is, the disclosed principles may obtain a better alcohol or other target clearance curve (absorption and clearance) that makes the disclosed alcohol bio sensor as accurate as current breath sensors or more accurate. According to the disclosed principles, alcohol or other targets may be measured using ethanol in the wearer's sweat. In addition, because the disclosed device uses sweat as surrogate for the BAC determination, and the fact that there is a lag in time before alcohol goes from the wearer's blood to sweat, the disclosed principles may include an algorithm for calibrating the user's sweat.

Motion (e.g., arm movement) and electrical activity in sweat can affect signals being monitored in traditional wearable sensors (i.e., wrong signals are monitored). An aim of the disclosed principles may be to measure the number of alcohol or other target molecules per second e.g., through flow from the body (blood to sweat) to transdermal (skin) and finally to vapors that contact the disclosed biosensor. According to the disclosed principles, a novel membrane apparatus between the device and skin may help reduce the problem of trans-dermal barrier (kinetics), meaning that the disclosed embodiments can better measure sweat and obtain better signals from the wearer.

A wearable patch to assess and monitor alcohol or other target via sweat, and then send the secure wireless data to a HIPAA-compliant mobile health cloud system has the potential to improve the lives of individuals with alcohol use disorders or other issues. Not only will this advance the knowledge of these disorders, but it can make the general public more aware of their limits for alcohol consumption during their daily lives. Additionally, the creation of a device to profile alcohol with a transdermal sensor may allow for a better clinical understanding of alcoholism than possible with current methods of biomonitoring.

Aspects of these systems may utilize one or more of the sensors, sensor housings, and/or sensor inlets disclosed herein.

While various embodiments have been described above, it should be understood that they have been presented by way of example and not limitation. It will be apparent to persons skilled in the relevant art(s) that various changes in form and detail can be made therein without departing from the spirit and scope. In fact, after reading the above description, it will be apparent to one skilled in the relevant art(s) how to implement alternative embodiments.

In addition, it should be understood that any figures which highlight the functionality and advantages are presented for example purposes only. The disclosed methodology and system are each sufficiently flexible and configurable such that they may be utilized in ways other than that shown.

Although the term “at least one” may often be used in the specification, claims and drawings, the terms “a”, “an”, “the”, “said”, etc. also signify “at least one” or “the at least one” in the specification, claims and drawings.

Finally, it is the applicant's intent that only claims that include the express language “means for” or “step for” be interpreted under 35 U.S.C. 112(f). Claims that do not expressly include the phrase “means for” or “step for” are not to be interpreted under 35 U.S.C. 112(f).

What is claimed is:

1. A sensor assembly comprising:
a housing;
a sensor mounted in the housing, the sensor including a sensing element adapted to detect a target and generate an electrical signal in response to detecting the target; and
an open cavity positioned to expose the sensing element to a fluid, the open cavity comprising a continuous distal perimeter adapted to define a substantially closed volume when the continuous distal perimeter is contacted with a surface.

2. The sensor assembly as recited in claim 1, wherein the open cavity comprises at least one sidewall.

3. The sensor assembly as recited in claim 2, wherein the at least one sidewall comprises at least one of a linear sidewall and a curvilinear sidewall.

4. The sensor assembly as recited in claim 3, wherein the at least one sidewall comprises a linear sidewall, and the linear sidewall comprises an angle of inclination.

5. The sensor assembly as recited in claim 4, wherein the angle of inclination ranges from 30 degrees to 60 degrees.

6. The sensor assembly as recited in claim 1, wherein the target comprises at least one of a substance and a condition.

7. The sensor assembly as recited in claim 1, where the continuous distal perimeter comprises a width dimension at least 20% greater than a width dimension of the sensing element.

8. The sensor assembly as recited in claim 1, wherein the target comprises an alcohol.

9. The sensor assembly as recited in claim 8, wherein the sensor comprises a fuel-cell-type alcohol sensor.

10. The sensor assembly as recited in claim 1, wherein the housing comprises a flexible material.

11. The sensor assembly as recited in claim 1, wherein the continuous distal perimeter of the open cavity comprises an elastomeric material.

12. The sensor assembly as recited in claim ii, wherein the elastomeric material comprises a polydimethylsiloxane [PDMS].

13. The sensor assembly as recited in claim 1, wherein the open cavity is remote from the housing.

14. The sensor assembly as recited in claim 1, wherein the assembly further comprises a retaining device adapted to retain the housing to the surface.

15. The sensor assembly as recited in claim 14, wherein the retaining device comprises one or more straps.

16. A method for detecting a target in a fluid, the method comprising:
contacting a sensor assembly with a surface of a body of a subject, the sensor assembly comprising a housing, a sensor including a sensing element mounted in the housing, and an open cavity positioned to expose the sensing element to a fluid and comprising a continuous distal perimeter, wherein the continuous distal perimeter of the open cavity defines a substantially closed volume with the surface of the body of the subject;
capturing at least some fluid emitted by the surface of the body of the subject in the substantially closed volume;
detecting, with the sensing element, a target in the fluid in the substantially closed volume; and
outputting, from the sensor, an electrical signal in response to detecting the target.

17. The method as recited in claim 16, wherein the target comprises at least one of a substance and a condition.

18. The method as recited in claim 16, wherein contacting the sensor assembly with the surface of the body of a subject further comprises providing a substantially fluid-tight seal between the continuous distal perimeter of the open cavity and the surface of the body.

19. The method as recited in claim 16, wherein outputting from the sensor the electrical signal comprises wirelessly transmitting the electrical signal to an external receiver.

20. An alcohol sensor assembly comprising:
a housing;
an alcohol sensor mounted in the housing, the alcohol sensor including a sensing element adapted to detect an alcohol and generate an electrical signal in response to detecting the alcohol; and
an open cavity positioned to expose the sensing element to a fluid, the open cavity comprising a continuous distal perimeter adapted to define a substantially closed volume when the continuous distal perimeter is contacted with a surface.

21. The alcohol sensor assembly as recited in claim 20, wherein the open cavity comprises at least one sidewall.

22. The alcohol sensor assembly as recited in claim 21, wherein the at least one sidewall comprises at least one of a linear sidewall and a curvilinear sidewall.

23. The alcohol sensor assembly as recited in claim 21, wherein the at least one sidewall comprises a linear sidewall, and the linear sidewall comprises an angle of inclination ranging from 30 degrees to 60 degrees.
